(19) United States
(12) Patent Application Publication
(10) Pub. No.: US 2024/0091272 A1
(43) Pub. Date: Mar. 21, 2024

(54) ENGINEERED MULTICELLULAR ORGANISMS

(71) Applicant: Trustees of Tufts College, Medford, MA (US)

(72) Inventors: Michael Levin, Beverly, MA (US); Gizem Gumuskaya, Medford, MA (US)

(21) Appl. No.: 18/254,929

(22) PCT Filed: Nov. 30, 2021
(86) PCT No.: PCT/US21/61222

§ 371 Date: May 30, 2023

Related U.S. Application Data
(60) Provisional application No. 63/119,517, filed on Nov. 30, 2020.

(51) Int. Cl. A61K 35/42; A61K 45/06; A61P 17/02; C12N 5/071; G01N 33/53

(52) U.S. Cl. CPC A61K 35/42; A61K 45/06; A61P 17/02; C12N 5/0688; G01N 33/53; G01N 2333/705

(57) ABSTRACT

Disclosed are engineered multicellular organisms. Also disclosed are systems and methods for designing, preparing, and utilizing the engineered multicellular organisms including methods of modulating tissue formulation and healing.

### CROSS-REFERENCE TO RELATED PATENT APPLICATIONS

The present application claims the benefit of priority under 35 U.S.C. § 119(e) to U.S. Provisional Application No. 63/119,517, filed on Nov. 30, 2020, the content of which is incorporated herein by reference in its entirety.

### BACKGROUND AND SUMMARY

The field of the invention relates to engineered multicellular organisms and systems and methods for designing, preparing, and utilizing engineered multicellular organisms. The engineered multicellular organisms may be configured for movement and other physical, computational, and biological activities.

Here, we introduce “Anthrobots,” which are spheroid- or ellipsoid-shaped multicellular biological robots (“biobots”) with a diameter ranging from 100 to 500 microns. Anthrobots have a built-in capacity for motility in aqueous environments based on locomotive appendages called “cilia” which cover their surface. Anthrobots are ciliated cells which may be derived from progenitor cells of ciliated epithelium, such as progenitor cells of human lung epithelium. Provided that Anthrobots are given the appropriate environmental conditions, Anthrobots self-organize into motile biological machines capable of moving in various trajectories including loops, straight lines, large arcs, tracking along features in their environment (living or inert), and even in zigzag patterns with a linear speed ranges from 15-200 microns/second.

Because Anthrobots develop on their own without the need for external manipulation or micromanagement, many of them can be grown in parallel. This makes Anthrobots amenable to easy mass fabrication, which not only makes their production more scalable and economical, but also enables easy generation of Anthrobots swarms that may collectively accomplish task that cannot be accomplished by a single Anthrobot.

Furthermore, Anthrobots can be loaded with exogenous payloads. Therefore, Anthrobots can be programmed on demand to execute a diverse set of tasks in different environments, including the human body. Because Anthrobots' base cell stock is derived from adult human tissue, as opposed to embryos or other species, Anthrobots can be personalized for each patient, enabling safe in vivo deployment of Anthrobots in the human body without causing inflammation or triggering an immune response. Once inoculated in the body via minimally invasive methods such as injection, various application can be imagined, including but not limited to, clearing plaque build-up in the arteries of atherosclerosis patients, bulldozing the excess mucus from the airways of cystic fibrosis patients, and locally delivering high doses of drugs of interest in target tissues. Anthrobots also can be used to modulate tissue formation in vivo and in vitro (e.g., for where the growth of tissue is modulated or sculpted by Anthrobots prior to transplantation into patients).

Finally, Anthrobots can be utilized to study cellular assembly in the formation of tissues and organs. The information gained from studies of cellular assembly can be utilized when administering regenerative medicine to a subject in need thereof. In particular, Anthrobots may be engineered to modulate tissue and organ formation in a subject in need of regenerative medicine.

### BRIEF DESCRIPTION OF THE FIGURES

FIG. 1. Illustration of strategy and results for obtaining apical-out aggregates of ciliated cells (i.e., any cells possessing motile cilia such as normal human bronchial epithelial cells (NHBE), or cells being able to be induced to develop motile cilia). A. Ciliated cells are explanted and cultured in a media comprising an extracellular matrix (ECM) for two weeks in which apical-in aggregates form. After which, the ECM is removed and the aggregates are further cultured and form apical-out aggregates having ciliated surfaces. B. Aggregates were immotile on day 0 post-removal of the ECM. C. After one week in culture in the absence of the ECM, the aggregates demonstrate motility. D. Staining illustrates conversio or similar cells (possessing motile cilia, or being able to be induced to develop motile cilia)n from apical-in aggregate to apical-out aggregate after 7 days.

FIG. 2. Localization of ciliated cells to the spheroid surface may be caused by a polarity reversal event. A. Motility launch profile post-culture in ECM-free media. B. Staining illustrates conversion from apical-in aggregate to apical-out aggregate over a 7 day period. C. Characterization of aggregates based on surface ciliation.

FIG. 3. Anthrobots have distinct movement trajectories. A. Data analysis of Anthrobot movement. B. Classification of Anthrobots based on movement, Class 1: straight movers; Class 2: loopers; Class 3: transition class; and Class 4: idles. C. Box plot describing each class per different movement metrics, showing polarizing distributions between classes. D. Absolute value for the angular speed illustrating a bimodal distribution. E. Distribution between the directionality of loopers illustrating that circular Anthrobots have a similar likelihood of moving clockwise or counterclockwise. F. Analysis of the stability of distinct states and possibility of state transitions using a Markov model, which shows stabilities ranging from 46.79% to 89.64%.

FIG. 4. Anthrobots can express exogenous proteins. A DNA vector encoding a constitutively expressed red fluorescent protein (RFP) was integrated at the single cell stage. These RFP-integrated cell populations were differentiated per our usual protocol as in FIG. 1, FIG. 2, and FIG. 3. Single cells were able to grow and differentiate into multicellular Anthrobots yielding fully fluorescent bot. Furthermore, because Anthrobot growth is monoclonal, a uniform distribution of RFP-integrated cells was observed.

FIG. 5. Time-lapse demonstrating anthrobots traversing a tear in tissue.

FIG. 6. Analysis of time-lapse data tracking anthrobots traversal of a tear in tissue.

FIG. 7. Placement of aggregate of anthrobots (i.e., a “superb”) on a scarred live tissue immediately after its placement on day 0, as well as on subsequent days of day 1 and day 2.

FIG. 8. Growth of native tissue after inoculation of superbot into tissue tear.

FIG. 9. Superbot promotion of a connection between the two sides of tissue at the site of superbot inoculation in the form of a “stitch.”

### DETAILED DESCRIPTION

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown, but are to be accorded the widest scope consistent with the principles and features disclosed herein. The following detailed description is to be read with reference to the figures. The figures, which are not necessarily to scale, depict selected embodiments and are not intended to limit the scope of embodiments of the disclosure. Skilled artisans will recognize the examples provided herein have many useful alternatives and fall within the scope of embodiments of the disclosure.

### Definitions and Terminology

Disclosed are engineered multicellular organisms and systems and methods designing, preparing, and utilizing the engineered multicellular organisms. The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a cell” should be interpreted to mean “one or more cells.” As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Engineered Multicellular Organisms

Disclosed are engineered multicellular organisms. Also disclosed are systems and methods for designing, preparing, and utilizing the engineered multicellular organisms.

The engineered multicellular organisms typically comprise an aggregate of cells. The aggregate of cells may comprise one or more different cell types. In some embodiments, the aggregate of cells comprises, consists essentially of, or consists of epithelia cells, such as ciliated epithelia cells. Suitable ciliated cells may include, but are not limited to, ciliated cells of the epithelial bronchial tissue of lungs. The organisms may comprise, consist essentially of, or consist of apical-out aggregates of ciliated cells.

Optionally, the engineered multicellular organisms may meet at least one of the following criteria: (i) the organism comprises less than about 1000 total cells, or less than about 900, 700, 600, 500, 400, 300, 200, or 100 cells (or the organism comprises a number of cells within a range bounded by any of these values (e.g., 100-1000 cells); and (ii) the organism has an effective diameter of less than about 2 mm, or less than about 1.5 mm, 1.0 mm, 0.9 mm, 0.8 mm, 0.7 mm, 0.6 mm, 0.5 mm, 0.4 mm, 0.3 mm, 0.2 mm, or 0.1 mm (or the organism has an effective diameter within a size range bounded by any of these values (e.g., 0.1-0.5 mm).

The engineered multicellular organisms preferably are self-motile and move when the cilia of the organisms are actuated by self-actuation. In some embodiments, the cilia of the organisms may be self-actuated. In other embodiments, the cilia of the organisms are actuated by external stimulus which may include but is not limited to electrical stimulation or optogenetics where the cilia have been genetically modified to express light-sensitive ion channels.

In some embodiments, the engineered multicellular organisms are in contact with a surface and move, for example linearly, when the cilia of the organisms are actuated. Preferably, the organisms move at a rate of at least about 1, 2, 3, 4, 5, 10, 15, 20, 25, 30, 35, 40, 45, 50 microns/second or faster when the cilia of the organisms are actuated. In some embodiments, the organisms are self-motile.

Preferably, the engineered multicellular organism have a life-span when placed in an physiologically suitable environment of at least about 3, 4, 5, 6, 7, 8, 9, 10, 11, 12, 13, 14, 15, 16, 17, 18, 19, 20, 21, 22, 23, 24, 25, 26, 27, 28, 29, or 30 days.

The engineered multicellular organisms comprise an aggregate of cells which may be referred to as a plurality of living cells that are cohered to one another. The aggregate of cells forms a three-dimensional shape.

The aggregate of cells of the disclosed organism may comprise one or more different cell types. The cell types of the aggregate may vary depending on the desired shape of the aggregate and/or function of the aggregate. Suitable cell types may include, but are not limited to, ciliated cells such as ciliated epithelia cells. Suitable ciliated cells may include, but are not limited to ciliated cells of epithelial bronchial tissue of the lungs.

Suitable cells may comprise animal cells. Suitable animal cells may human cells.

The aggregate of cells may comprise, consist essentially of, or consist of ciliated cells. In some embodiments, the aggregate of cells may comprise or may not comprise additional non-ciliated cell types.

In some embodiments, the engineered multicellular organisms are non-innervated and/or or non-cartilaginous. The multicellular organisms may be described as “engineered” because they are different from naturally occurring organism that arise without the guidance of human ingenuity and modifications. In other words, the multicellular organisms are synthetic and non-naturally occurring, albeit the multicellular organism may utilize endogenous cell:cell signaling and morphogenesis.

The aggregate of cells of the engineered multicellular organisms may comprise cells that have been engineered to express a heterologous molecule. In some embodiments, the cells of the organisms are engineered to express a heterologous protein or secrete specific desired molecules.

In embodiments in which the aggregate of cells comprises cells that have been engineered to express a heterologous molecule, suitable heterologous molecules that are expressed may include therapeutic agents. Other suitable heterologous molecules may include enzymes that metabolize a target substrate, which may include toxins. Other suitable heterologous molecules may include receptors for a target ligand (e.g., a target ligand sensed by the organism), or sensors of light, heat, and other physical properties in the environment.

Preferably, the engineered multicellular organisms are self-repairing. In some embodiments, if the aggregate of cells is subjected to deaggregation (e.g., physical damage that disrupts aggregation of the cells), the cells will reaggregate to re-form the aggregate of cells.

The engineered multicellular organisms may be configured in order to perform tasks. In some embodiments, the organism is configured for moving a target object (e.g., by pushing a target object). In further embodiments, the organism is configured for moving target objects (e.g., by pushing target objects) and collecting the moved target objections (i.e., aggregating the target objects).

The engineered multicellular organisms may be configured to have a cavity. In some embodiments, the engineered multicellular organisms are configured to have a cavity for capturing and/or transporting a target object.

The engineered multicellular organism may be utilized in a number of applications. In some embodiments, the organisms are utilized in methods for delivering a therapeutic agent to a subject in need thereof, where the method comprises engineering the organisms o to express the therapeutic agent and administering the organism to the subject.

In other embodiments, the engineered multicellular organisms are utilized in methods for removing a target substrate from an environment (e.g., a toxin from an environment). The methods may comprise engineering the organisms to express an enzyme that metabolizes the target substrate and placing the organism in the environment to remove the target substrate from the environment.

In other embodiments, the engineered multicellular organism are utilized in methods for detecting a target ligand in a sample. The methods may comprise engineering the organisms to express a receptor for the target ligand and place the organism in the sample, where the organism generates a signal after the receptor binds the target ligand.

### EXAMPLES

The following examples are illustrative and should not be interpreted to limit the scope of the claimed subject matter.

### Example 1. Anthrobots: Human-Derived Self-Organizing Biological Robots

Abstract

Control of biological structure and function is a key need for regenerative medicine and synthetic biology. While organoids have been made from many cell types, these have heretofore lacked functionality. Similarly, existing “biobots” (with the exception of our previous Xenobot invention) have largely been highly engineered constructs, with cells seeded on precise scaffolds which afford only one kind of capability, and required external actuation. Together, this strongly constrains the possible uses of this technology. Our Anthrobots are novel synthetic living machines which 1) are made of cells that do not require (although could include) genetic editing, 2) can be made of any human's cells thus enabling use in patient bodies without immune rejection, 3) exhibit self-organization that is difficult to achieve by bioengineering, and 4) exhibit motile, self-driven behavior which suggests numerous uses in intelligent sensing, cellular repair/sentinels, and drug delivery. Such biological robots have many advantages over currently much larger electro-mechanical robots.

Introduction

Here we introduce Anthrobots, spheroid-shaped multicellular biological robots (biobots) with diameters ranging from 100 to 500 microns. Anthrobots have a built-in capacity for motility in aqueous environments thanks to the locomotive appendages called cilia covering their surface. These multicellular biobots start out as single cells, derived from the human lung, and within three weeks—given the appropriate environmental conditions—they self-organize into motile biological machines capable of moving in various trajectories including loops, straight lines, large arches, and even in zigzag patterns with a linear speed ranging from 5-50 microns/second.

In the last decade, interest in developing biobots, which often come in the form of motile biogenic assemblies, have seen a rapid surge(1,2). Early examples 2A of biobots are hybrid devices comprised of biological cells supported by inert chemical substances such as gels or 3D-printed scaffolds(3-7). Through constitutive or inducible contractions of these biological cells, which are often derived from myocytes, entire structure displaces in space and time, hence the term biobot.

Main drawback of these existing hybrid biobots are their dependence on external form giving machinery such as 3D printers or molds. This is a drawback because such external dependence inherently limits economical mass-fabrication of these biobots. A more advanced biobots introduced earlier this year, called the Xenobots, featured a fully cellularized body, independent of an inert scaffold supporting the cells(8). These Xenobots can be shaped by hand and are able to move on their own. As a result, they are one step ahead of the race for economical mass-fabrication; however, they are still limited because they nevertheless need to be manufactured one by one. Because Anthrobots develop on their own without the need for external manipulation or micromanagement, many of them can be grown in parallel. This grants our method with the unique capacity for easy mass fabrication, which not only makes their production more scalable and economical; but it also enables easy generation of Anthrobot swarms which may collectively accomplish tasks that cannot be accomplished by a single Anthrobot.

Furthermore, unlike Xenobots which are derived from frog embryos, Anthrobots are derived from adult human tissue, therefore can be personalized for each patient, enabling safe in-vivo deployment of these robots in the human body without causing inflammation or triggering an immune response. Once inoculated in the body via minimally invasive methods such as injection, various applications can be imagined, including but not limited to clearing plaque buildup in the arteries of atherosclerosis patients, bulldozing the excess mucus from the airways of cystic fibrosis patients, and locally delivering high doses of drugs of interest in target tissues.

Results

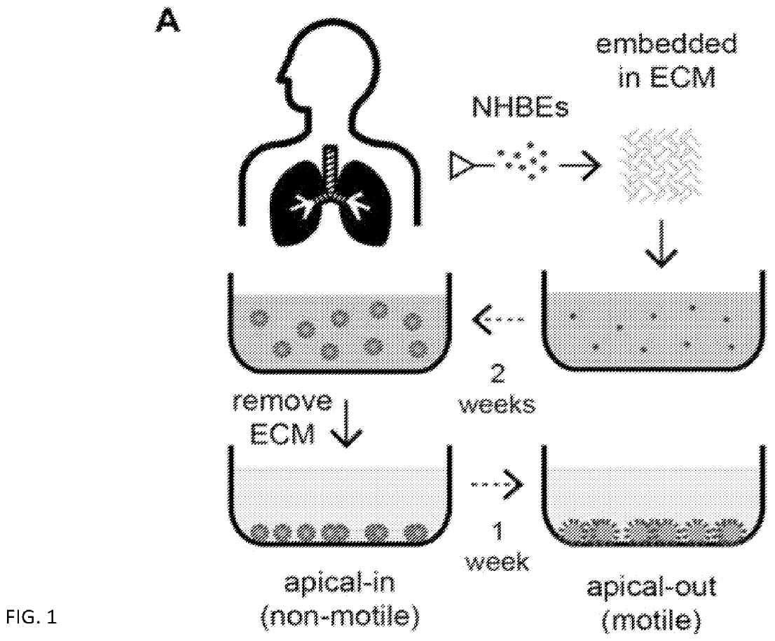

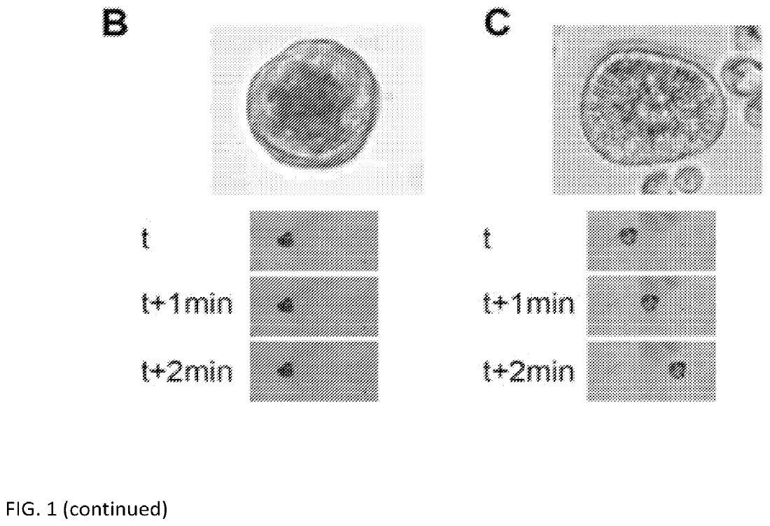

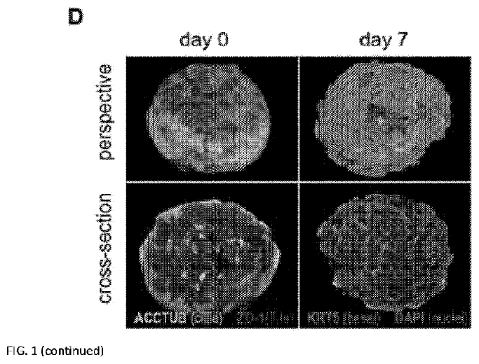

Human bronchial epithelial cells self-construct into motile biobots, i.e, Anthrobots. It has been established in the literature that normal human bronchial epithelial cells (NHBE) have the capacity to form airway organoids, which are multicellular spheroids with multiciliated cells lining their lumen in an apical-in configuration(9). For the purposes of developing motile multicellular structures, we hypothesized that localization of such multiciliated cells on the spheroid surface (i.e., the cortex) in an apical-out configuration may grant the structure with a locomotive ability. Because it has previously been shown in the literature that transferring apical-in organoids from a dense matrix environment to a lighter hydrophobic environment enables them to polarity switch and arrive at an apical-out configuration(10), we experimented with a similar approach (FIG. 1A). To test this the possibility of a similar morphological reorganization event in the context of airway organoids, we first formed the airway organoids by culturing NHBE cells in a three-dimensional matrix environment. When organoids matured, we dissolved the surrounding matrix and transferred the organoids into a low-adhesion environment on day 0. At this point, airway organoids were completely immotile (FIG. 1). However, within a week of culture in this hydrophobic environment, spheroids started showing a significant increase in their motility (FIG. 1C), becoming three dimensional motile structures that we call Anthrobots.

Anthrobot motility results from ciliated cells emerging on the cortex. Because cilia are known to function as locomotive appendages, we hypothesized that this significant increase in motility is attributable to the emergence of cilia on the spheroid cortex following dissolution of the surrounding matrix. To test this hypothesis, we fixed and stained both the immotile day 0 and the highly motile day 10 biobots with several polarity markers, including the cilia marker acetylated tubulin (FIG. 1C). In this experiment, we consistently observed significantly higher levels of cilia localization on the cortex of the day 10 bots compared to day 0 bots. Based on these observations we conclude that airway organoids, when removed from surrounding matrix and placed in a low-adhesive environment, undergo major morphological reorganization, resulting in multiciliated cells to localize on the cortex. This reorganization renders airway organoids constitutively motile for extended durations, without the need for any external input or induction.

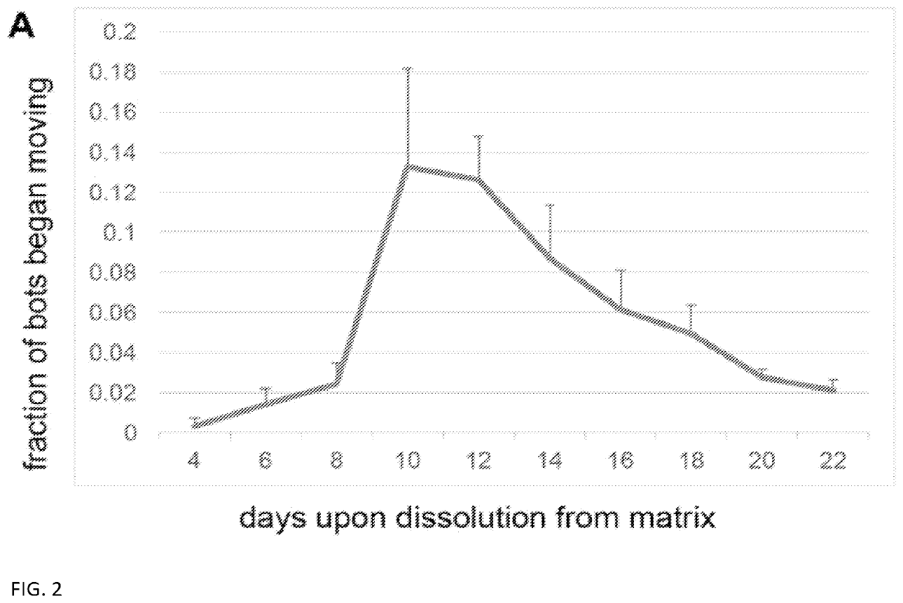

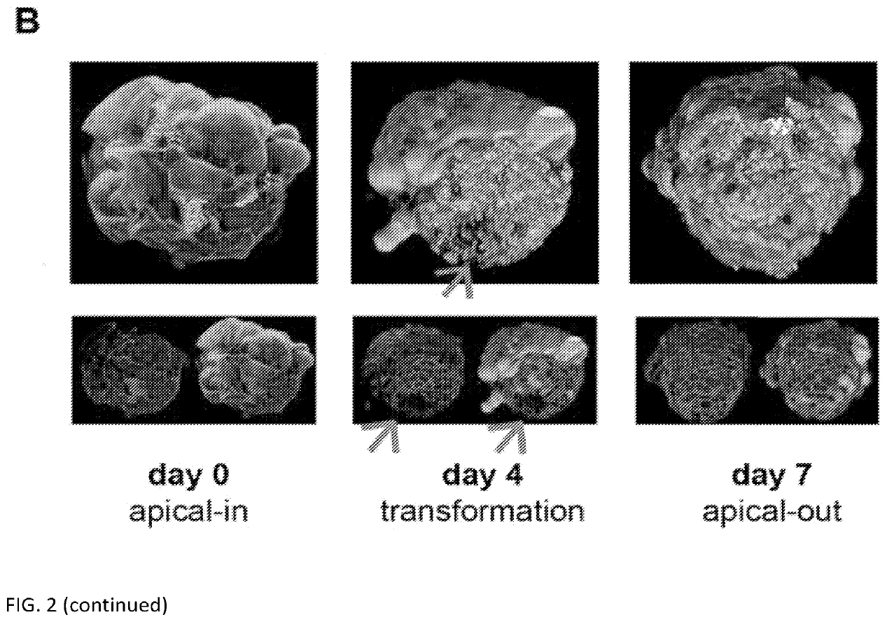

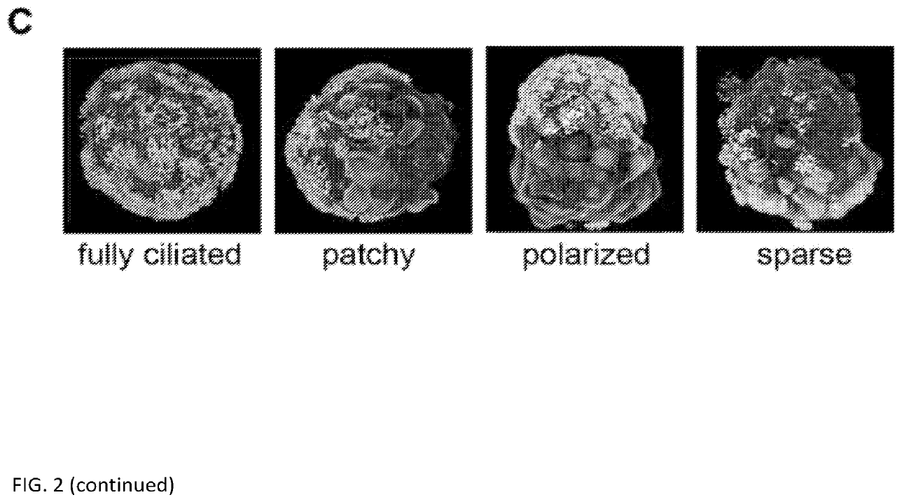

Localization of ciliated cells to the spheroid surface may be caused by a polarity reversal event. In order to better understand the spatiotemporal dynamics of this morphological reorganization event enabling cilia to localize to the spheroid surface, we first counted the number of spheroids that became motile on a bidaily basis across a 3 weeks time frame. This experiment showed a non-linear motility launch profile with a peak on day 10 (FIG. 2A), suggesting a significant change in morphology within the first 10 days upon dissolution from the matrix. In order to examine the spatiotemporal dynamics this morphological reorganization event, we fixed spheroids at different time points within the first 10 days (FIG. 2B). On day 0, as expected, we observed a ciliated lumen lined with tight junctions and an undifferentiated basal cell population covering the cortex. On days 3-7, we repeatedly observed the formation of an invaginating hole on the spheroid surface, bridging luminal space to the cortex. Furthermore, we consistently observed a ciliated region around this invagination, indicating a potential migration event of multiciliated cells from the luminal space to the cortex. After all, it is known that across an interface with differential density on either side, ciliated cells polarize towards the more fluid medium as seen in native epithelial tissues of the airways, ovaries, and brain ventricles. Accordingly, in this context, the surrounding lesser density environment generated by the low-adhesive properties of the culture dish may be promoting the luminal multiciliated cells to migrate towards the surface of the sphere, causing organoid to partially or entirely polarity switch. Aligned with this hypothesis, on days 8-10, we persistently observed ciliated extrusions on spheroid surfaces, enabling motility. Throughout our analyses, we have not detected the presence of primary cilium, which is known to precede the formation of multiciliated cells. Therefore, we do not have any evidence for further differentiation of the basal cell population into multiciliated cells, which would be an alternative hypothesis explaining the emergence of cilia on spheroid surface. This alternative hypothesis must be tested with transcriptomic analyses for further rejection or verification as that might reveal further insights that cannot be obtained with immunological assays.

During our immunological assay, we have also realized that although the cortical morphology of the day 0 non-mover spheroids was mostly uniform, this was not the case for the motile day 10 bots. To further investigate motile bots' cortical structure, especially as it relates to cilia distribution, we collected a pool of motile bot samples from days 11, 18, and 25 and repeated the immunological study using above markers. As a result of this experiment, we observed that motile spheroids can assume several distinct morphologies (FIG. 2C), which might explain the diverse movement trajectories they exhibit.

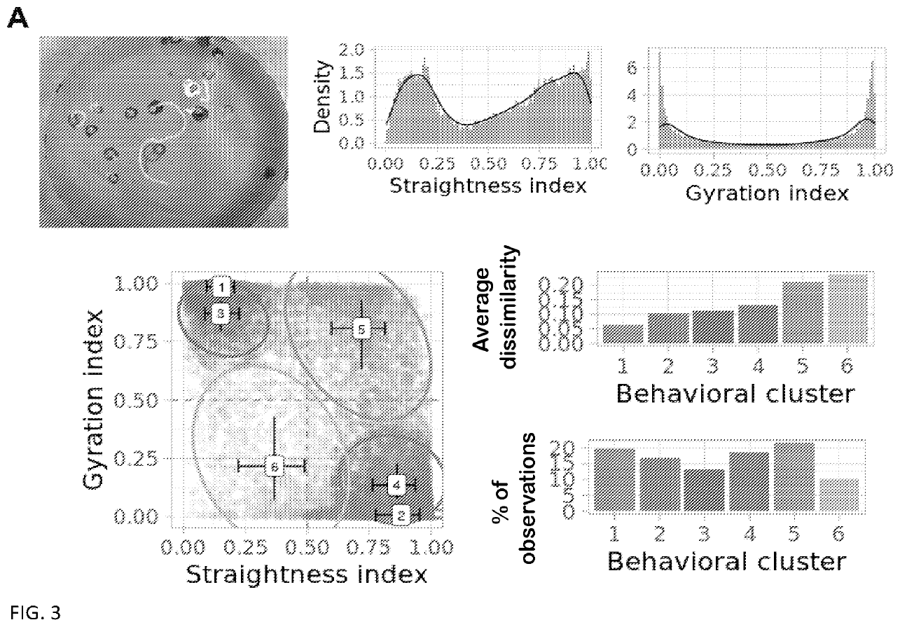

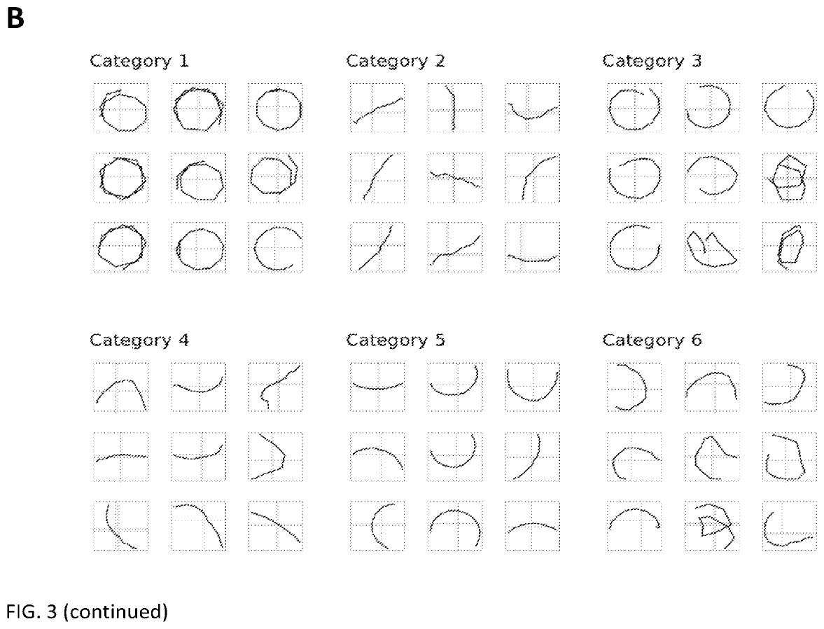

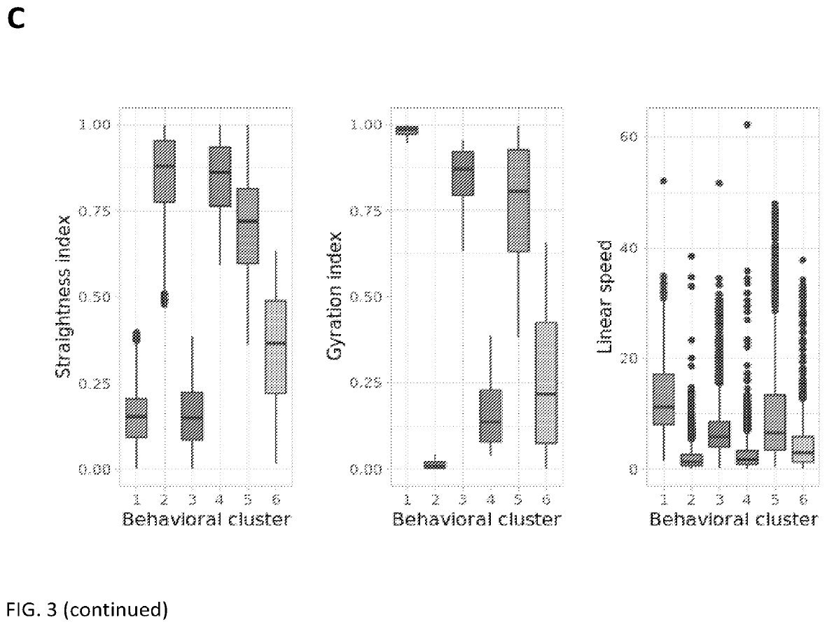

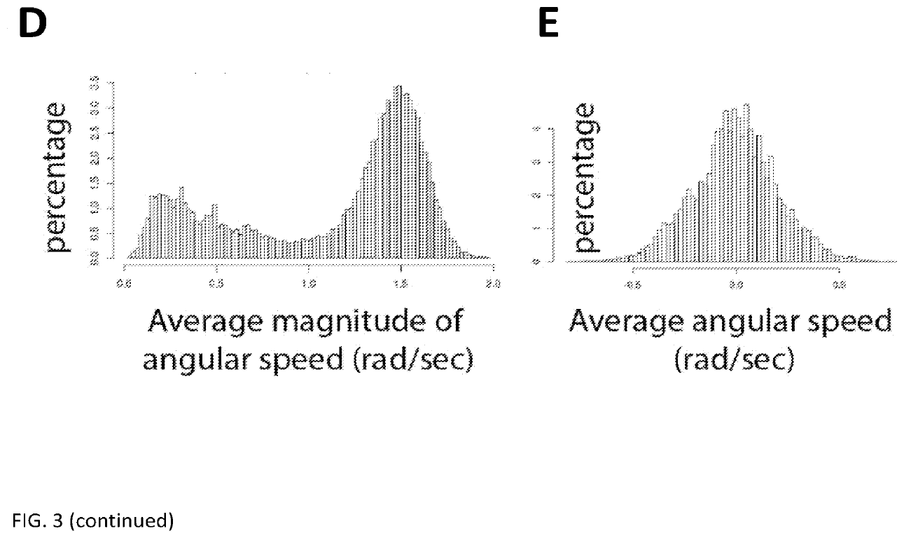

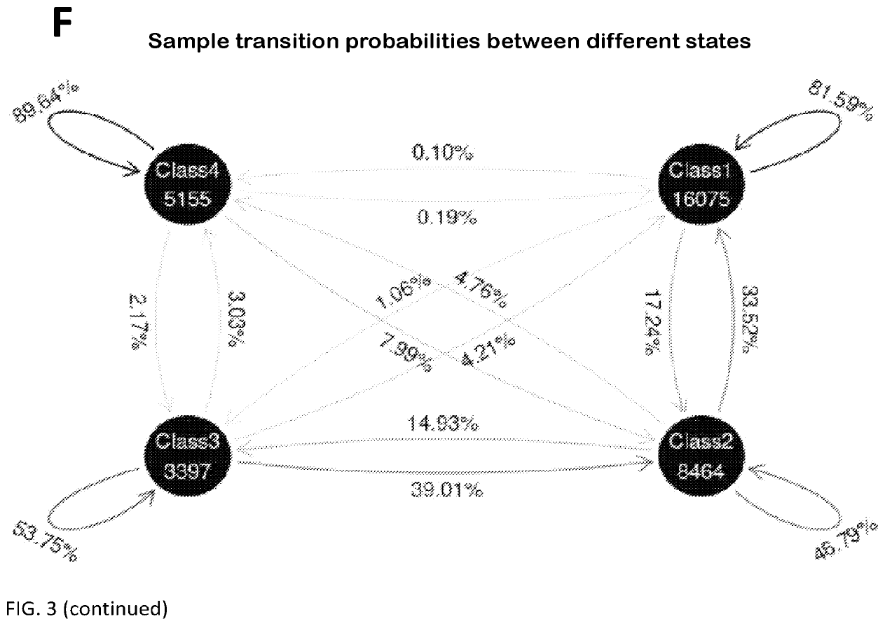

Anthrobots have distinct movement trajectories. During our observations, we noticed that Anthrobots move in various distinct trajectories including loops, arches, and straight lines. To further analyze these patterns, we first collected timelapse videos of around 200 bots in groups of 4-8 for an average of 10 hours per group. We have then tracked these groups to obtain trajectory coordinates for individual bots, which were further clustered into four distinct classes using PCA analysis (FIG. 3A AND FIG. 3B). Each class represents a distinct phenotypic movement pattern: straight movers, loopers, a transition class, and idles. FIG. 3B shows a box plot describing each class per different movement metrics, showing polarizing distributions between classes. Another distinct distribution indicating the presence of a specialized class is emerged when we quantified the angular speeds of all bots. Absolute value for the angular speed showed a bimodal distribution (FIG. 3C), indicating the presence of a distinct loopers class. Furthermore, this analysis showed that, when we account for the sign of angular speed, there is a uniform distribution between the directionality of loopers (FIG. 3D), meaning circular Anthrobots have a similar likelihood of moving clockwise or counterclockwise. Finally, we analyzed the stability of these distinct states and possibility of state transitions using a Markov model (FIG. 3E), which showed stabilities ranging from 46.79% to 89.64%. From this movement analysis, we conclude that Anthrobots have distinct movement trajectories with a possibility of switching in between them.

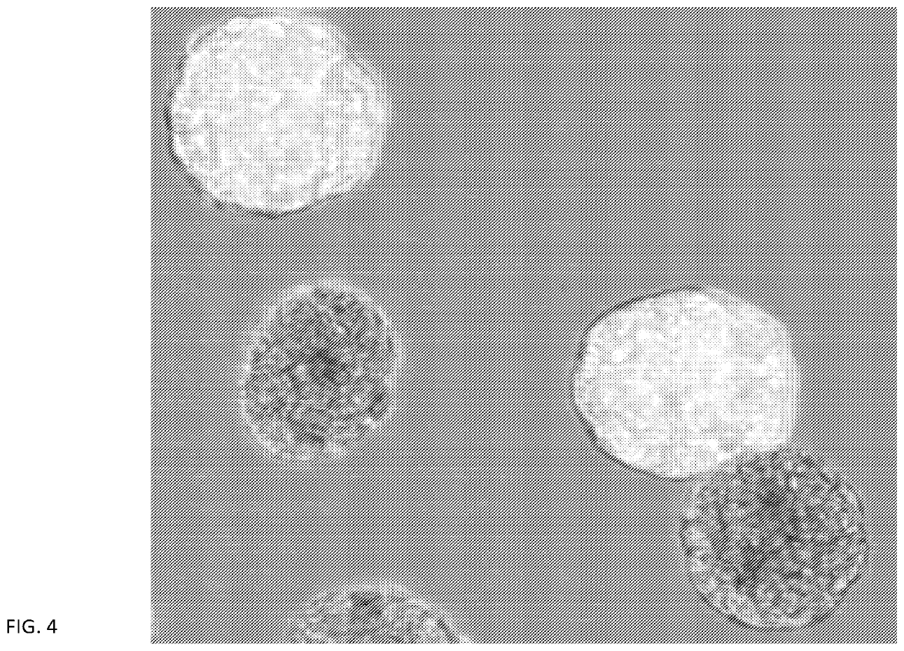

Anthrobots can be engineered using exogenous vectors. Anthrobots already have many emergent features such as multicellularity, motility, and reasonable consistency of their spheroidal shape. In order to further expand their capabilities in a programmable fashion, we experimented with engineering them using exogenous vectors. As the first step towards this goal, we integrated a DNA vector encoding a constitutively expressed red fluorescent protein at the single cell stage. We have then differentiated these RFP-integrated cell populations per our usual protocol. Single cells were able to grow and differentiate into multicellular Anthrobots without any issues, yielding fully fluorescent bots. (See FIG. 4). Furthermore, because Anthrobot growth is monoclonal, we observed a uniform distribution of RFP-integrated cells in our engineered bots. This demonstrates that these Anthrobots can express foreign DNA, which will include ion channels, adhesion molecules, receptors, enzymes, planar polarity proteins, signaling pathways, and synthetic proteins.

### CONCLUSION

We demonstrate a method to produce self-organizing motile synthetic living machines. We envision a variety of uses in the human body and for in vitro bioengineering applications to sculpt tissue, catch rogue cells, deliver compounds, etc. Most importantly, these bots can take advantage of the metabolic, computational, and sensory capacities of real cells, which means they can be programmed (via transgenes from synthetic biology toolkits, via nanomaterials and microenvironments, and via electro-mechanical-optical stimulation) for rich, intelligent behaviors.

### REFERENCES

1. Ricotti L, Trimmer B, Feinberg A W, Raman R, Parker K K, Bashir R, et al. Biohybrid actuators for robotics: A review of devices actuated by living cells. Sci Robot. 2017 Nov. 29; 2(12):eaaq0495.
2. Menciassi A, Takeuchi S, Kamm R D. Biohybrid systems: Borrowing from nature to make better machines. APL Bioeng. 2020 Jun. 1; 4(2):020401.
3. Sakar M S, Neal D, Boudou T, Borochin M A, Li Y, Weiss R, et al.

Formation and optogenetic control of engineered 3D skeletal muscle bioactuators. Lab Chip. 2012; 12(23):4976.
4. Chan V, Park K, Collens M B, Kong H, Saif T A, Bashir R. Development of Miniaturized Walking Biological Machines. Sci Rep. 2012 December; 2(1):857.
5. Nawroth J C, Lee H, Feinberg A W, Ripplinger C M, McCain M L, Grosberg A, et al. A tissue-engineered jellyfish with biomimetic propulsion. Nat Biotechnol. 2012 August; 30(8):792-7.
6. Raman R, Cvetkovic C, Uzel S G M, Platt R J, Sengupta P, Kamm R D, et al. Optogenetic skeletal muscle-powered adaptive biological machines. Proc Natl Acad Sci. 2016 Mar. 29; 113(13):3497-502.
7. Park S-J, Gazzola M, Park K S, Park S, Di Santo V, Blevins E L, et al. Phototactic guidance of a tissue-engineered soft-robotic ray. Science. 2016 Jul. 8; 353(6295):158-62.
8. Kriegman S, Blackiston D, Levin M, Bongard J. A scalable pipeline for designing reconfigurable organisms. Proc Natl Acad Sci. 2020 Jan. 28; 117(4):1853-9.
9. Hild M, Jaffe A B. Production of 3-D Airway Organoids From Primary Human Airway Basal Cells and Their Use in High-Throughput Screening. Curr Protoc Stem Cell Biol [Internet]. 2016 May [cited 2020 Oct. 21]; 37(1). Available from: https://onlinelibrary.wiley.com/doi/abs/10.1002/cpcs.1
10. Co J Y, Margalef-Catala M, Li X, Mah A T, Kuo C J, Monack D M, et al. Controlling Epithelial Polarity: A Human Enteroid Model for Host-Pathogen Interactions. Cell Rep. 2019 February; 26(9):2509-2520.e4.

### Example 2. Anthrobots can Traverse Live Tissues

One of the major benefits of anthrobots being derived from human cells (bronchial epithelial cells) that when they are prepared with patient's own cells and inoculated back into that patient (i.e., when the anthrobots are autologous), the anthrobots should not trigger an immune response as they would be recognized as a part of “self.” This should enable anthrobots to perform certain tasks within the body without the need for a surgical manipulation of the tissue.

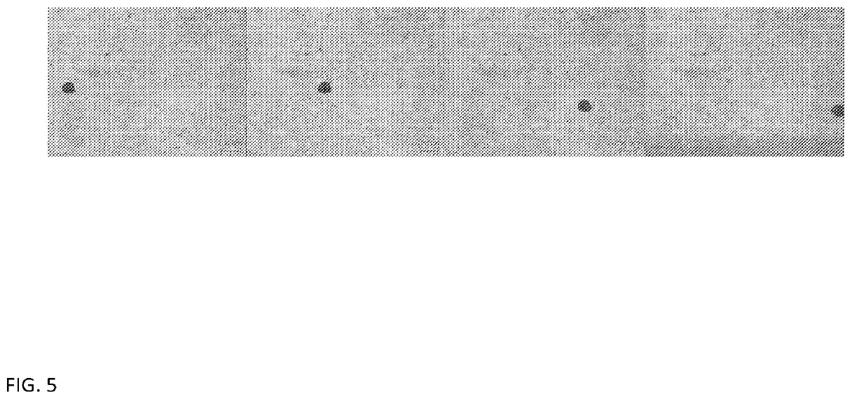

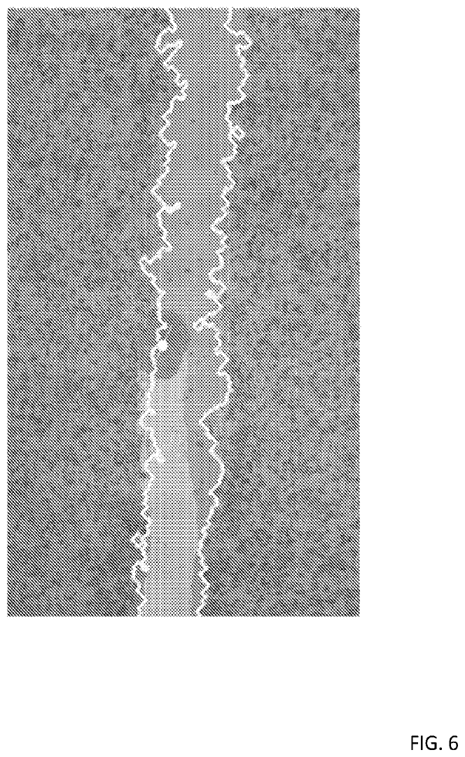

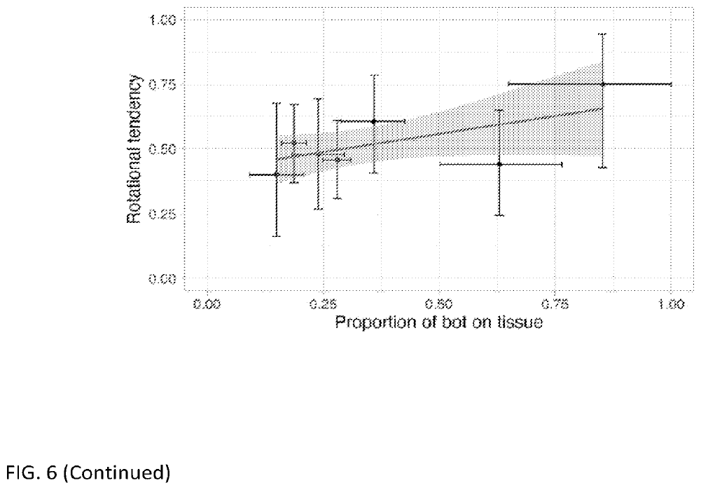

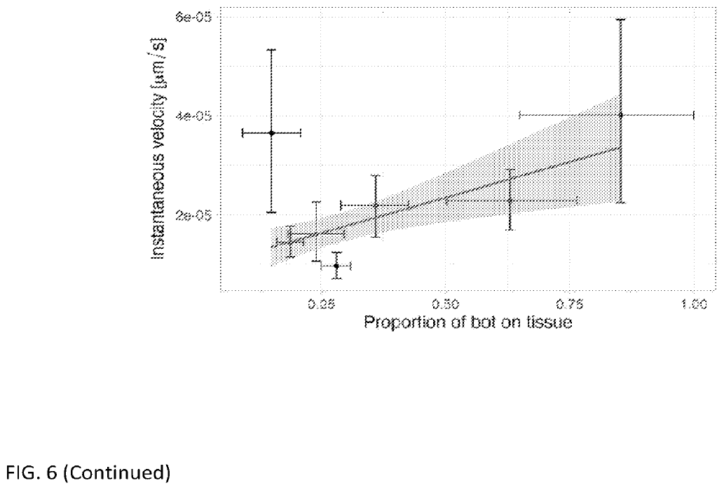

In order to test the premise that anthrobots can traverse narrow spaces within live tissues, we prepared hiNSC-derived neuronal tissues and made tears in these otherwise intact tissues as a proxy for internal tissue tears in the body. When we inoculated anthrobots to these narrow tissue tears, we observed that several high-speed bots traversed these tears with ease. (See FIG. 5). When we analyzed this traversal time-lapse data by way of tracking the anthrobots as they traverse the scar and calculating their heading as well as area covered, we found out that anthrobots that have a higher rotational tendency and or a higher speed end up “exploring” the tear better by covering a higher percentage of the tear interface. (See FIG. 6).

Therefore, we conclude that anthrobots have the ability to traverse long stretches of tissue tears, which often occur deep internal tissues within a patient's body, access to which would otherwise require a surgical intervention.

### Example 3. Anthrobots can Promote Healing in Live Tissue Tears

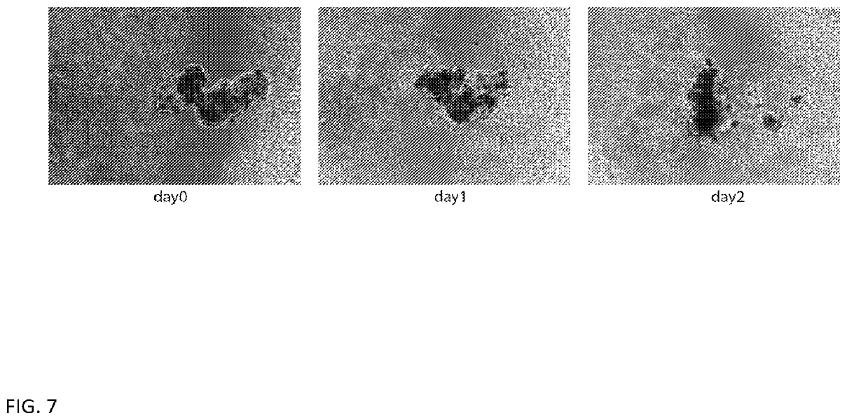

After having demonstrated that anthrobots can traverse scarred live tissues, next we decided to examine whether anthrobots can promote healing on these internal scars. In order to determine whether anthrobots can promote tissue healing, we constructed larger “superbots” by constraining multiple anthrobots in separate small dishes to promote their random aggregation with one another. We then carefully placed these superbots into arbitrary sites along the tissue tear in a way to cross the entire width of the tear and hence bridge the two sides of the damaged tissue. FIG. 7 shows a superbot on a scarred live tissue immediately after its placement on day 0, as well as on subsequent days of day 1 and day 2.

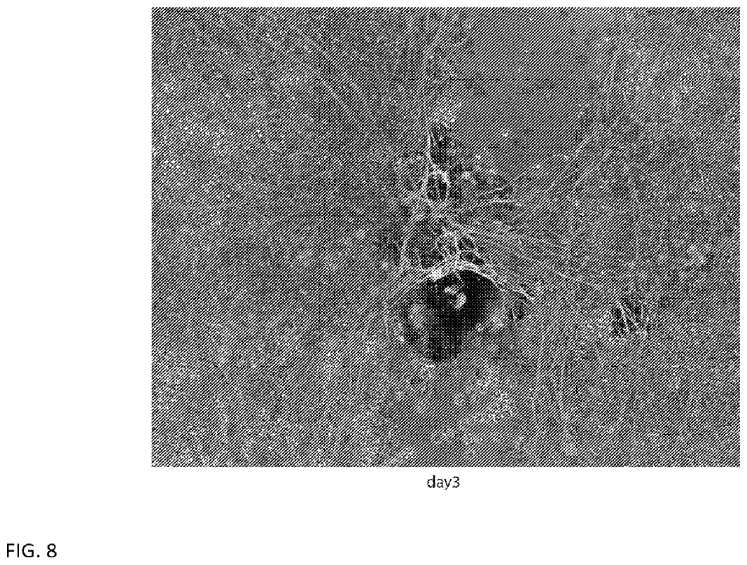

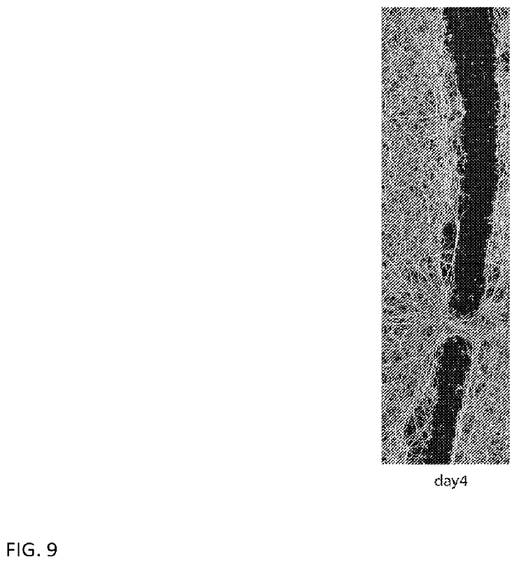

Using confocal microscopy, we observed that, within the next 72 hours upon inoculation of the superbot into the tissue tear, a substantial growth of the native tissue took place. (See FIG. 8). The superbot promoted a connection between the two sides of tissue at the site of superbot inoculation in the form of a “stitch.” (See FIG. 9).

Therefore, larger anthrobot aggregates can aid the healing of a live tissue and can be used to promote the healing of live tissues internally without requiring a surgical intervention.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An engineered multicellular organism comprising an apical-out aggregate of ciliated cells, wherein the organism is self-motile.

2. The organism of claim 1, wherein the ciliated cells are human cells.

3. The organism of claim 1, wherein the ciliated cells are epithelial cells.

4. The organism of claim 1, wherein the ciliated cells are normal human bronchial epithelial cells.

5. The organism of claim 1, wherein the organism consists of biological material and/or does not comprise any inorganic material, for example as a scaffold.

6. The organism of claim 1, wherein the organism comprises a sensor for detecting a target molecule.

7. The organism of claim 1, wherein the cells of the organism self-assembly.

8. The organism of claim 1, wherein the cells of the organism move and assemble cells to form other organisms.

9. The organism of claim 1, wherein the organism has an effective diameter of about 100-500 microns.

10. The organism of claim 1, wherein the ciliated cells are normal human bronchial epithelial cells.

11. The organism of claim 1, wherein the ciliated cells are engineered to express a heterologous molecule.

12. The organism of claim 11, wherein the heterologous molecule is a therapeutic agent.

13. The organism of claim 11, wherein the heterologous molecule is an enzyme that metabolizes a target substrate.

14. The organism of claim 11, wherein the heterologous molecule is a receptor for a target ligand.

15. The organism of claim 1, wherein the aggregate of cells reaggregates after the aggregate is subjected to deaggregation.

16. The organism of claim 1 wherein the organism is configured for moving a target object, optionally wherein the organism comprises a hole or cavity for holding the target object.

17. The organism of claim 1, wherein the organism is configured to have a cavity for capturing and/or transporting a target object.

18. A plurality of the organism of claim 1, wherein the plurality exhibits collective and/or coordinated behavior.

19. The plurality of claim 18, wherein the collective and/or coordinated behavior is collective and/or coordinated movement.

20. A method for delivering a therapeutic agent to a subject in need thereof, the method comprising engineering the organism of claim 1 to express the therapeutic agent and administering the organism to the subject.

21. A method for removing a target substrate from an environment, the method comprising engineering the organism of claim 1 to express an enzyme that metabolizes the target substrate and placing the organism in the environment.

22. A method for detecting a target ligand in a sample, the method comprising engineering the organism of claim 1 to express a receptor for the target ligand and place the organism in the sample, wherein the organism generates a signal after the receptor binds the target ligand.

23. A method for modulating the formation of tissue and/or an organ in a subject in need thereof, the method comprising administering the organism of claim 1 to the subject, wherein the organism modulates the formation of the tissue and/or the organ in the subject, optionally, wherein the organism forms part of the tissue and/or the organ in the subject.

24. A method for promoting healing of a tear in tissue in a subject in need thereof, the method comprising inoculating the tear with an aggregate of the organism of claim 1.

25. The method of claim 24, wherein the ciliated cells are obtained from the subject and/or the ciliated cells or organisms are autologous with respect to the subject.

26. A method for modulating the growth of tissue in vitro, the method comprising contacting the tissue with an aggregate of the organism of claim 1.
